(19) United States
(12) Patent Application Publication
(10) Pub. No.: US 2020/0008890 A1
(43) Pub. Date: Jan. 9, 2020

(54) COUPLING FOR A ROBOTIC SURGICAL INSTRUMENT

(71) Applicant: IMPERIAL INNOVATIONS LIMITED, London (GB)

(72) Inventors: CARLO ALBERTO SENECI, LONDON (GB); JIANZHONG SHANG, LONDON (GB); GUANG-ZHONG YANG, LONDON (GB)

(21) Appl. No.: 16/338,099

(22) PCT Filed: Oct. 4, 2017
(86) PCT No.: PCT/EP2017/075255

§ 371 Date: Apr. 10, 2019

(30) Foreign Application Priority Data
Oct. 4, 2016 (GB) 1616827.0
Mar. 30, 2017 (GB) 1705094.9

(51) Int. Cl. A61B 34/30; A61B 34/00; A61B 90/00

(52) U.S. Cl. CPC A61B 34/30; A61B 34/71; A61B 2017/00477; A61B 90/06; A61B 34/76

(57) ABSTRACT

The present invention provides a load sensing device for a surgical robot comprising a load sensing means and a hook mounted to the load sensing means, wherein the coupling is slideable longitudinally and engageable with a tendon for actuating a surgical instrument such that longitudinal movement of the hook imparts a load on the tendon and the load sensing means measures such load.

### FIELD OF THE INVENTION

The present invention relates to a coupling for a robotic surgical instrument.

### BACKGROUND

In robotic surgery one of the most important features to have is a quick and reliable instrument interchange, in fact it allows surgeons to use more instruments specifically designed for each sub-tasks of a surgical procedure. Therefore having a quick and reliable system to do this becomes of large importance to reduce “dead time” in the surgical workflow.

In addition to this, another feature is of great importance: force feedback. The adoption of surgical robots has been increasing over the last few years, but so far it has always be a known issue the fact that current surgical robots are not provided with force feedback. This is mainly due to the fact that most of the work in this field focused on providing instruments with force sensors mounted at the tip.

This invention proposes a solution to both the problems stated above, by implementing linear couplings that have embedded force sensing, to measure the pulling force that the robot is applying to the tendons.

It is against this background that the present invention has arisen.

### SUMMARY OF THE INVENTION

One aspect of the invention provides means for quickly changing surgical tools during a surgical procedure to minimize “dead time” during surgery. In particular, an attachment interface between a surgical robot and a surgical instrument comprising: a first coupling slideably mounted to a surgical robot and a second coupling mounted to a robotic surgical instrument, wherein the first coupling is movable between a first position in which the first coupling engages the second coupling and prevents longitudinal movement of the robotic surgical instrument relative to the surgical robot and a second position in which the first coupling and the second coupling are disengaged permitting longitudinal movement of the robotic surgical instrument relative to the surgical robot.

Another aspect of the invention provides integrated force sensing into its actuation, in order to implement force feedback in a cost-effective way, by avoiding placing force sensors on the surgical instrument tip, which is a major barrier of sensing integration in current surgical robotics. In particular, a surgical robot comprising an attachment interface for actuation of a robotic surgical instrument attachable thereto, wherein the attachment interface comprises means for measuring pulling force applied by the surgical robot to the robotic surgical instrument.

Another aspect of the invention provides a safety system that measures the pulling force on each of a plurality of tendons. If a tendon breaks the pulling force would instantly reduce to zero and the system would immediately restrict further operation of the surgical robot. In particular, a load sensing device for a surgical robot comprising a plurality of load cells and an equal number of hooks, wherein measurement of tendon tension is transmitted from each respective load cell to a controller and the controller is configured to lock actuation of the surgical instrument upon measurement of a tendon tension indicative of a respective tendon break.

Another aspect of the invention provides a surgical tool that can be rotated continually around its longitudinal axis such that its rotational range is greater than 360°. In particular, a surgical robot comprising a body and a mounting interface, wherein the body is rotatable relative to the mounting interface.

Another aspect of the invention provides a robotic surgical instrument comprising a rigid hollow shaft positioned between a mounting hub and a surgical tool, wherein the mounting hub has a plurality of couplings slideably mounted thereto, wherein each coupling is associated with a respective tendon passing through the rigid hollow shaft and arranged between said coupling and the surgical tool.

Another aspect of the invention provides apparatus for robotic surgery comprising a surgical robot and a robotic surgical instrument, wherein the robotic surgical instrument comprises a surgical tool having at least two degrees of freedom of movement, each degree of freedom of movement being controlled by a respective tendon and wherein the surgical robot comprises at least two motors where each motor drives a respective tendon.

Another aspect of the invention provides a method of measuring force applied to a robotic surgical instrument, the method comprising: i) attaching a robotic surgical instrument having a plurality of tendons to a surgical robot using an attachment interface; ii) applying a pre-load to each tendon; iii) actuating the robotic surgical instrument using the tendons and maintaining the pre-load on all non-active tendons; and iv) measuring the pulling force applied to each active tendon.

Another aspect of the invention provides a surgical robot comprising a hollow cylindrical body mounting seven motors therein, wherein each motor is controlled by a respective motor control board provided on a motherboard, wherein each motor control board is modular. Another aspect of the invention provides apparatus for robotic surgery comprising a surgical robot and a robotic surgical instrument wherein the robotic surgical instrument comprises a near field communication chip for communicating with the surgical robot.

### FIGURES

The invention will now be described with reference to the following figures.

FIG. 1 shows a wristed surgical robot mounted on a six DoF serial manipulator for global positioning.

FIG. 2 shows an overview of the surgical robot with an instrument attached.

FIG. 3 shows a detailed view of the rotation mechanism of the instrument of FIG. 2.

FIG. 4 shows a detailed view of the linear actuators of the instrument of FIGS. 2 and 3.

FIG. 5 shows a detailed view of the slider nuts of the instrument of FIGS. 2 to 4.

FIG. 6 shows a detailed view of the instrument release mechanism of the instrument of FIGS. 2 to 5.

FIG. 7 shows an exemplary wristed surgical grasper as a complete unit (top) and exploded (bottom).

FIG. 8 shows a detailed view of the tip of the wristed surgical grasper of FIG. 7.

FIG. 9 shows an example experimental set up.

FIG. 10 shows a representation of the tendons cantilevers with respect to the rotation of the ais of the jaw and the wrist link.

FIG. 11 shows experimental results obtained from a first experiment.

FIGS. 12a and 12b show a control scheme for one embodiment of instrument joint.

FIG. 13 shows experimental results obtained from a second experiment.

FIG. 14 illustrates position control repeatability of instruments according to aspects of the invention.

### DESCRIPTION

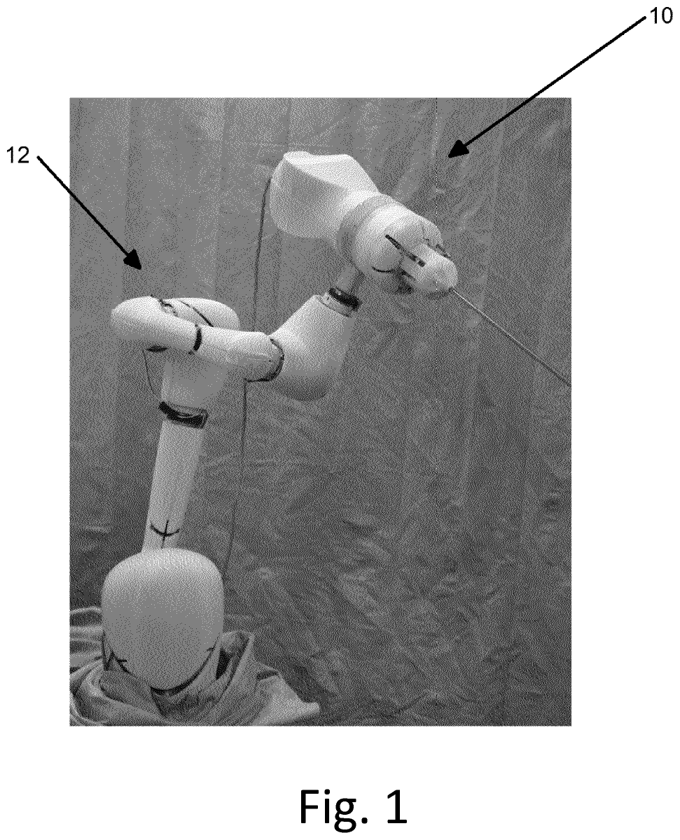

FIG. 1 shows a wristed surgical robot 10 mounted on a six Degree of Freedom (DoF) serial manipulator 12 for global positioning. The surgical robot 10 is configured to mount a wristed instrument, and is capable of being rapidly manufactured and assembled. The surgical robot 10 has also been designed to allow quick integration of new instruments through the use of a modular design. Additionally, the surgical robot 10 has integrated force sensing into its actuation, as will be described below, in order to implement force feedback in a cost-effective way, by avoiding the placement of force sensors on the surgical instrument tip. Such placement has been a major barrier of sensing integration in prior art surgical robots.

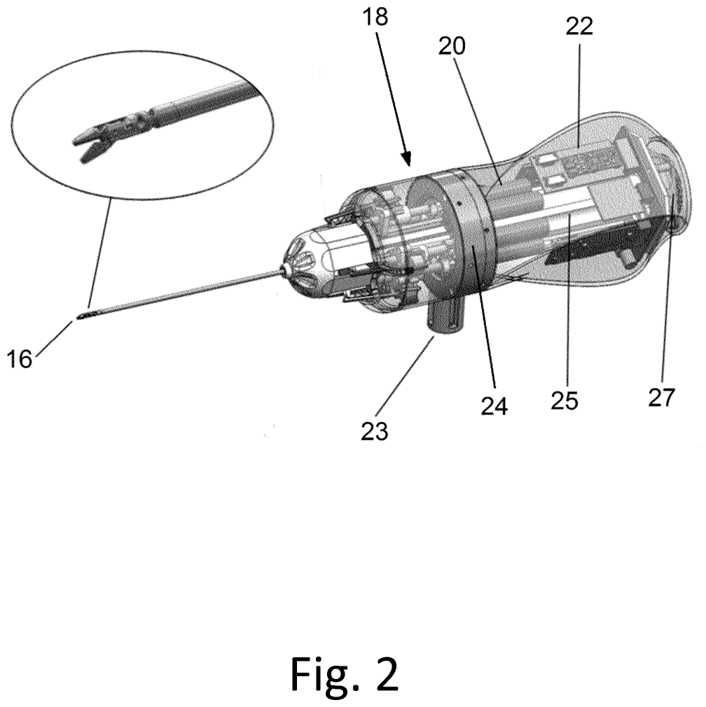

FIG. 2 shows an overview of the surgical robot 10 with an end-effector (in this embodiment, a wristed grasper) 16 attached. The surgical robot 10 shown in FIG. 2 is a modular attachment for the six DoF serial manipulator 12 shown in FIG. 1. The serial manipulator 12 provides the surgical robot 10 with global positioning and a Remote Centre of Motion (RCM), with two perpendicular axes of rotation and one of translation.

The surgical robot 10 is provided with three additional DoFs by the end-effector 16: two DoF wrist rotation and one DoF axial rotation. The surgical robot 10 comprises an instrument mounting interface with fast couplings to give the freedom to attach surgical tools, for example an end-effector 16, as shown in FIG. 2. The end-effector 16 is disposable and has a diameter of 3 mm, which is suitable for applications where the surgical site is characterized by narrow space.

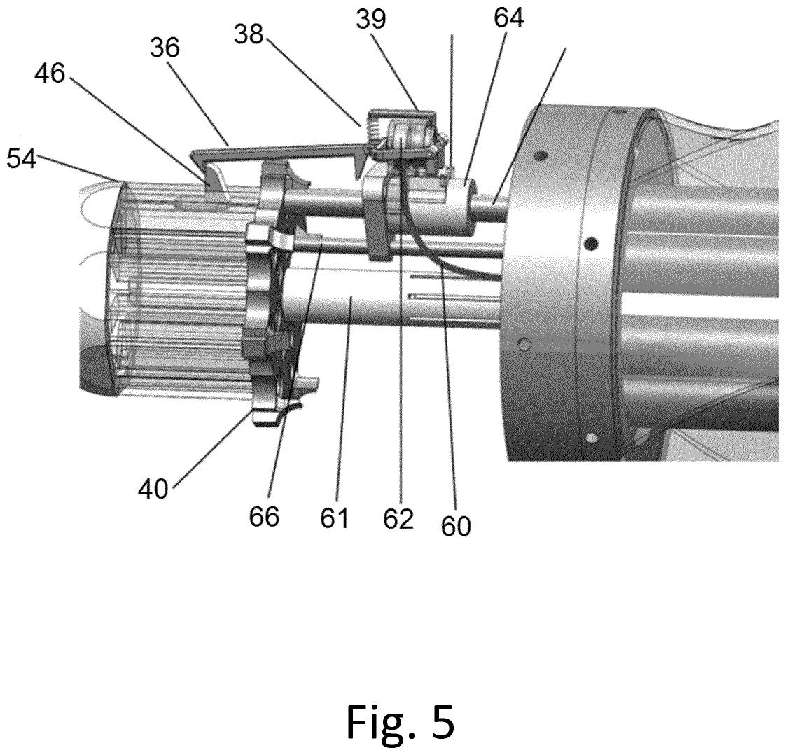

The surgical robot 10 comprises three pairs of antagonistic tendons (not shown in figures) to drive the end-effector 14. Instead of using three motors to drive the three pairs of tendons, as in most tendon driven systems, this device uses six actuators to drive the six tendons, which gives a redundant actuation, i.e. should an actuator fail, the actuator on the other tendon of the tendon pair can still be used to drive the tendon normally driven by the failed actuator. This arrangement, combined with the use of a load cell (62—see FIG. 5) on each tendon, or coupling, to monitor the tension of all tendons, allows for more precise instrument control than that achieved in prior art surgical robots while providing embedded force sensing.

When a surgical instrument is plugged onto the surgical robot 10, the surgical robot 10 performs an initializing step by pulling back each of the tendons until a set pretension (e.g. 2N) is achieved on each tendon. When this step has been performed the initial position may be identified and the surgical robot 10 can actuate the tendons, whilst maintaining the pretension, which compensates for possible backlash.

In other embodiments, a single motor can be used to drive a pair of tendons. However, redundant actuation is lost in this configuration.

The advantage of integrated force sensing also allows the application of the surgical robot 10 in areas where the instrument-tissue interaction is very delicate, for example in brain or fetal surgery.

The surgical robot 10 comprises a cylindrical body 18 which hosts all the main components of the robot, including the aforementioned motors 20 and driving electronics 22, as well as the actuation mechanisms and the fast couplings. In the embodiment shown, the seven motors 20 used for the surgical robot 10 are DC brushless Maxon EC 13 Ø13 mm 12W motors, although any suitable motor could be used in practice. Each motor is connected to a planetary gearhead with reduction ratio of 67:1. The motor driving electronics 22 is placed at the back of the motors 20, directly mounted on the main body of the robot 10 as shown. The driving electronics 22 contains both power circuitry and communication circuitry. The surgical robot 10 may be attached to the serial manipulator 12 through a connection post 23.

In the embodiment shown, the power provided to the surgical robot 10 is 24VDC and the communication protocol used is a customized RS-485 protocol running at 4 MBaud. The driving electronics 22 comprises a motherboard. The motherboard allocates eight slots for plugging in the motor controller boards and one slot for a voltage regulator board. The motherboard also hosts a connector for a multicore-shielded cable, which is used to transfer both power and communication signals between the surgical robot 10 and a host computer and power supply through an interface 27 on the rear of the surgical robot 10.

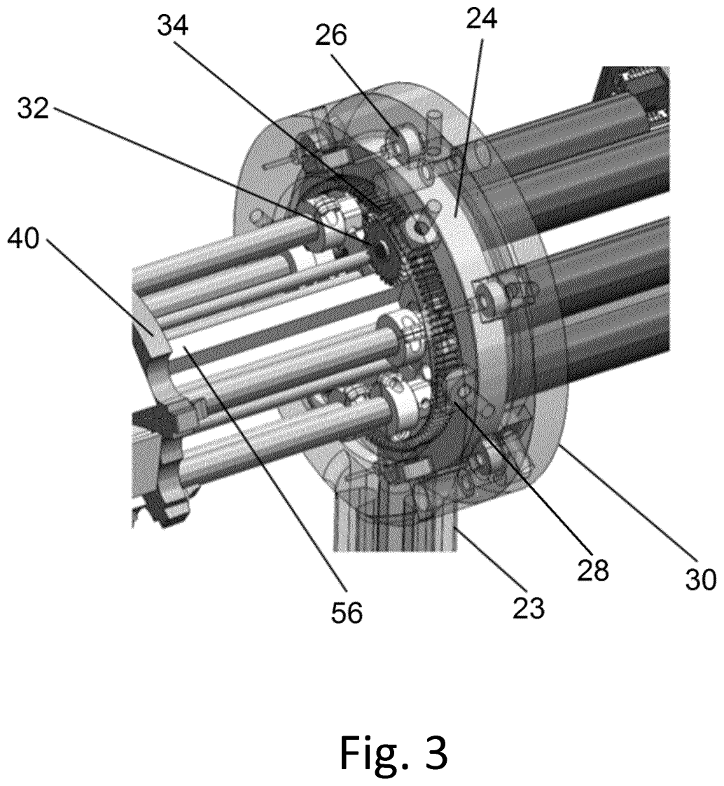

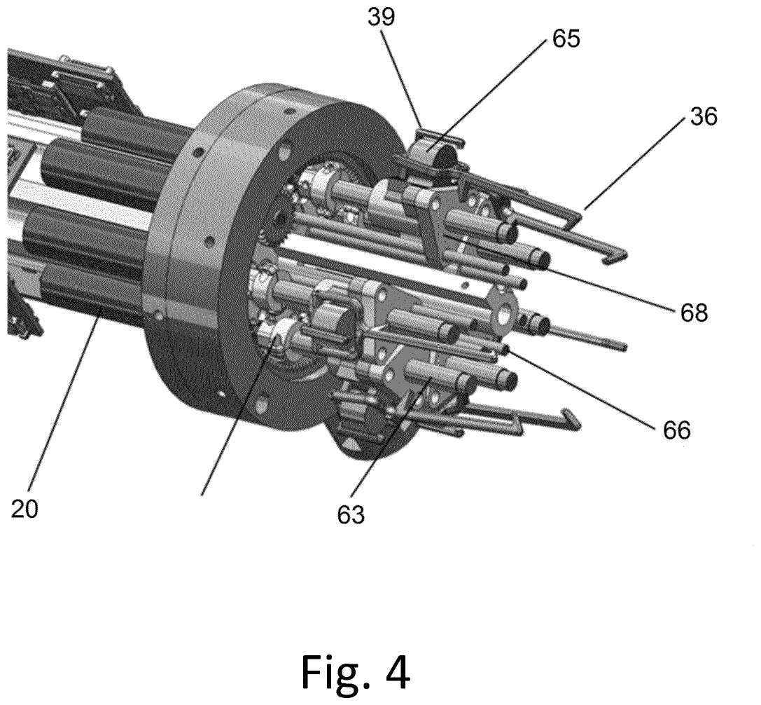

The main body 18 of the surgical robot 10 is provided with a one DoF rotation mechanism about its longitudinal axis. As shown in FIG. 3, an outer ring 24 provides an interface between the surgical robot 10 and the serial manipulator 12. It is designed to allow the main body 18 to rotate freely by 360°. It achieves this through the use of eighteen bearings, which allow for smooth rotation of the main body 18. Six of the 7×7×3 mm bearings are distributed around the main body 18 ring circumference (one such bearing is indicated at 26 in FIG. 3), while the remaining 12 are split between front and back side of the ring (one such bearing is indicated at 28 in FIG. 3), contained in the interface and a back plate 30. Rotation about the longitudinal axis of the surgical robot 10 is facilitated by the peripheral bearings 26, while axial translation is constrained by the bearings 28 placed at the front and at the back of the outer ring 24 of the main body 18.

Motion is transferred from a brushless motor to the main body 18 through a pinion-annular gear coupling 32, 34. In the embodiment shown, the pinion 32 has a reference diameter of 14 mm and module 0.5. The annular gear 34 is formed integrally with the interface, for example through machining, casting or 3D printing. This minimizes the amount of assembly work needed. In the embodiment shown, the annular gear 34 has a reference diameter of 56 mm and module 0.5, therefore the gear reduction ratio is 1/4. Other gear reduction ratios may be used in practice, as needed. As exemplary dimensions, in the embodiment shown the main body 18 has a maximum diameter of 88 mm and overall length of 240 mm.

The actuation of the end-effector 16, as mentioned above, relies on the use of six motors 20 that drive six 6 mm lead screws 63, with 1 mm lead and 59 mm long. The lead screws 63 are connected to the motors 20 through the use of flexible couplings to compensate for possible shaft misalignment. Each lead screw 63 carries a precision anti-backlash nut ActiveCAM (RTM) that allows precise movement with a very small drag torque. In addition, a polytetrafluoroethylene (PTFE) film is used to coat the screws 63 and reduce friction between screw 63 and nut 68. In the embodiment shown, the nuts 68 are 22.8 mm long and the screws 63 are 59 mm, which gives the nuts a linear Range Of Motion (ROM) of 36.2 mm. It is advantageous for the ROM to be configured to be larger than needed, as this maintains a higher degree of compatibility with customized instruments. Six 3 mm diameter stainless steel rods 66 are used to maintain the orientation of the nuts 68, preventing them from rotating with the screw 63. The rods 66 are fixed between the outer ring 24 of the main body 18 and a front plate 40.

The friction between the two components is very limited, as the rod 66 is formed of stainless steel and the nut is made of a hard and self-lubricated Acetal. Each lead screw nut 68 also carries a load cell holder 65. This is inserted into the cylindrical opening of the carrier 64, which also allows the sensor's lead cable 60 to exit from the side of the cavity. The lead cables 60 are then routed through the hollow front shaft 61 of the robot body 18, to the back 25 of the main body 18 of the surgical robot 10, and connected to the driving electronics 22. An example of a suitable load cell 62 for use with the invention is a Futek LLB130—FSH02950, which has a cylindrical shape with Ø9.5 mm and thickness 3.3 mm. The maximum load measurable is 222N, which is sufficiently large for the tendons used in a surgical instrument attached to the surgical robot 10.

A pressing element is also inserted into the load cell holder 65 and in contact with the load cell 62. The pressing element transmits a pulling force from the slider nut to a slider hook 36, which transmits the force to a surgical instrument attached to the surgical robot 10. Although a slider hook is shown, it will be appreciated that any other suitable coupling means could be used. This arrangement gives a direct connection between the load cell 62 and the tendons of an attached instrument. As the tendons are practically aligned at the instrument proximal end, this simplifies the force measurement.

The pressing element is provided with a hinge where the slider hook 36 can be attached. A spring 38 (attached between the slider hook 36 and a post 39) with Internal Diameter (ID) of 2.3 mm and Outside Diameter (OD) of 3 mm and a rate of 77 N/mm is used to maintain the slider hooks 36 engaged with sliding couplings of a surgical instrument attached to the surgical robot 10.

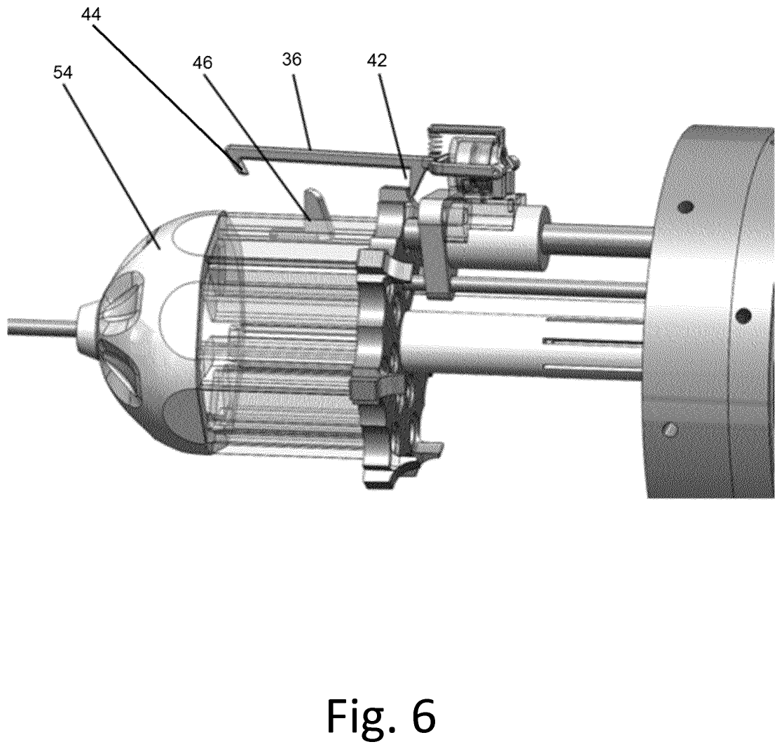

When the slider nuts 68 are advanced to their furthest forward position a portion of the slider hook 36 engages with a cam-feature on a rear surface of the surgical robot front plate 40. This rotates the slider hook 36 to a disengaged position, as shown in FIG. 6, which enables automatic instrument release. The slider hook 36 presents a contact element 42 to the cam-feature to apply a load to the slider hook 36, which culminates in a torque that lifts the slider hook 36 from an instrument's sliding coupling 46, automatically releasing the surgical instrument.

The sliding coupling 46 can be engaged by the sliding hook 36 in either a pushing or pulling manner, depending on application. Where the sliding hook 36 pushes on the sliding coupling 46, it is urged towards the robotic wristed instrument 16. Where the sliding hook 36 pulls on the sliding coupling 46, it is urged away from the robotic wristed instrument 16.

All of the surgical robot's components except from the motors 20, lead screws, nuts and bearings, are produced with rapid manufacturing techniques. The plastic components are made of a photopolymer cured with UV light and with similar mechanical properties to acrylonitrile butadiene styrene (ABS). The metal components have been produced with Selective Laser Melting (SLM) of stainless steel 316.

The sliding hook 36 can be actuated by way of a variety of actuation means. The illustrated embodiments show a lead screw configuration. It will be appreciated that other actuation means such as a rack and pinion or hydraulic cylinder and piston could also be used. The illustrated embodiments show a load sensor that is independent of the motors 20 but it will be appreciated that the load sensor could be an integral part of the sensors to measure current as a direct correlation of load.

The robotic wristed instrument 16 is advantageously simple in design and assembly. One of the drawbacks of additive manufacturing, especially when dealing with metal SLM, is that often components need a certain degree of post processing, for instance, to remove the support structure. In order to reduce the effect that this has on the full exploitation of rapid prototyping advantages, the surgical robot 10 was designed with the objective of reducing the overall number of components and simplifying the assembly procedure. Generally speaking, the unit cost of additive manufacturing is higher than the one obtained with mass production in industrial processes. However, it is a cost-effective way of manufacturing at low volume, and can achieve functionality and complexity that traditional manufacturing process cannot achieve.

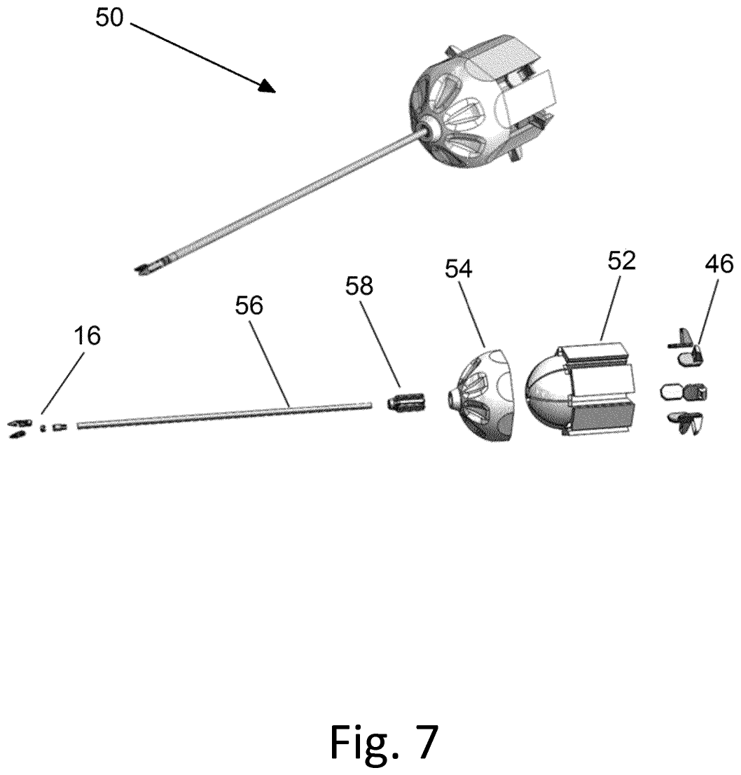

As shown in FIG. 7, the surgical instrument 50 described herein is constructed of only fourteen components, excluding the driving tendons. Due to the degree of simplicity, each surgical instrument 50 only requires about twenty minutes of assembly time per instrument.

Therefore, simplifying the assembly through the use of as few components as possible also contributes to reducing the unit cost, by reducing the labour needed to complete the assembly task. In addition, having a limited unit cost allows to making the surgical instrument 50 disposable, further reducing the complexity of the design and manufacture, since there is no need to implement solutions for re-sterilization.

The surgical instrument 50 comprises an instrument proximal base 52 and base cover 54, which are produced with rapid prototyping, using a Fused Deposition Modeling (FDM) printer with ABS as the material used. The surgical instrument further comprises an instrument shaft 56, which is a stainless steel tube with outer diameter 3 mm and inner diameter 2.5 mm. The components of the end effector 16, the tendons separator 58 and the sliding couplings 46 are manufactured with SLM of stainless steel 316. The instrument's sliding couplings 46 are actuated by the robot's sliding hooks 36, which engage on the instruments couplings 46, after the surgical instrument 50 is inserted and the slider hooks 36 are moved backwards.

Stainless steel tendons are inserted in the sliding couplings 36 and crimped to prevent the tendons from escaping. In the embodiment shown, the tendons chosen have diameter 0.35 mm and strand 7×7. The breaking load of these tendons is approximately 80N, which is sufficient for the application devised for this surgical instrument.

The six tendons run from the sliding couplings 46 towards the three DoF end-effector, to actuate it as three pairs of antagonistic tendons. The tendons pass through a groove that is obtained on a dome-shaped distal part of the instrument's base 52 and in the internal part of its cover 54. The groove acts as a guide keeping the tendons path constant and providing a relatively low friction plastic-metal interface for the tendons. The six tendons each enter the tendon separator 58 at their respective places and are routed together towards the instrument's end effector 16, passing through the rigid hollow shaft 56. The tendon separator 58 is not provided with pulleys, which again simplifies the construction. Although this design results in the tendons rubbing against the metal structure of the separator 58, the instrument is designed to be disposable. This means that the amount of friction deterioration experienced by the instrument will be negligible over the time for which the instrument is used, and so the instrument's performance will not be adversely affected.

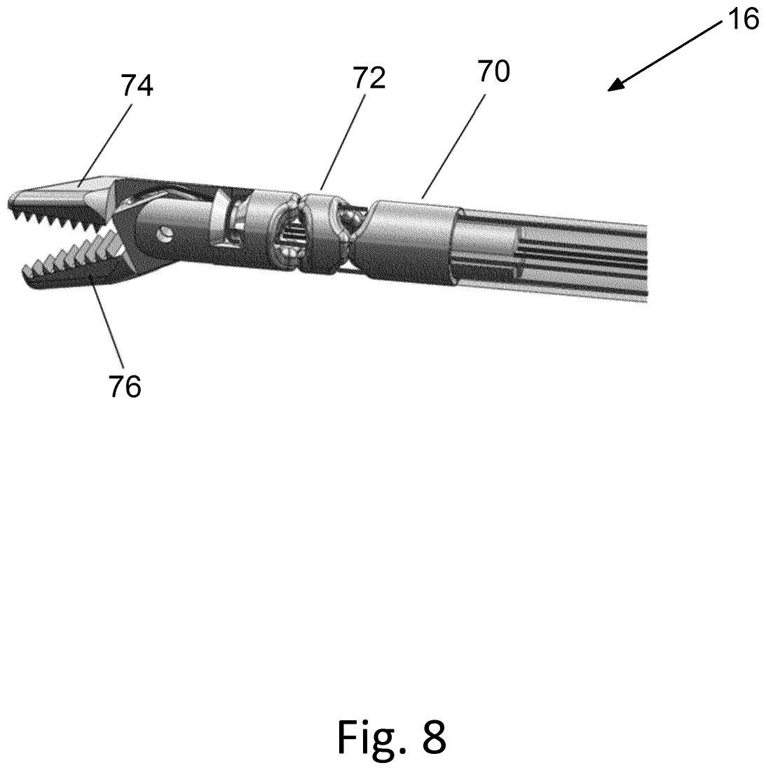

FIG. 8 discloses a detailed view of the end-effector 16, which in the shown embodiment comprises a wristed grasper. The wristed grasper comprises a shaft 70 and wrist 72 which carries a pair of opposed jaws 74, 76 The range of motion of the wrist is ±60° in both perpendicular planes and the grasper's jaw can open such that there is an angle of 90° between the jaws 74, 76, so that the wristed grasper can behave both as a grasper and as a dissector. The pair of tendons that actuates the grasper's jaw pass through a central hole in the wrist 72, in order to reduce the coupling effect. One of the advantages of this design is that it is possible to redesign the tip of the surgical instrument 50 and its functionality, and easily integrate it to the surgical robot 10, by keeping the same instrument-robot coupling interface.

Experimental Data

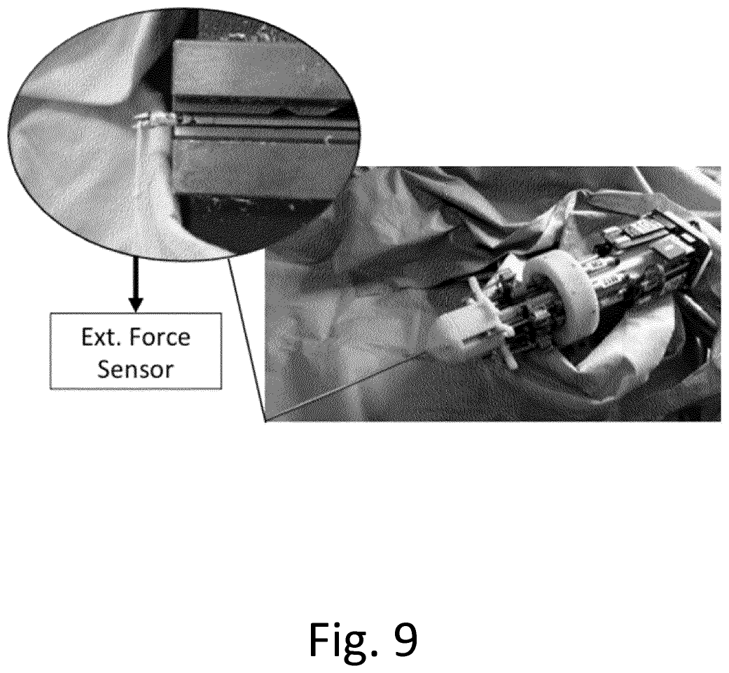

A number of experiments have been performed to validate the capability of the surgical robot 10, and to measure the interaction forces between the surgical instrument 50 and the environment. The CY8CKIT-050 development board from Cypress Semiconductor was used to acquire data from the six load cells 62 installed on the surgical robot 10. On the board, a PSoC5LP (Programmable System-on-Chip) implemented signal conditioning, amplification, and digitization. The data was sent to a host computer via USB communication. The set-up of these experiments includes the surgical robot 10 with its wristed surgical instrument 50 and load cells 62. An additional external force gauge was used for the sole purpose of calibration and validation (Sauter FK250). This last one was grounded and fixed with respect to the instrument's rigid shaft 56, to avoid bias in the force reading at the tip of the instrument 50, due to possible rigid shaft 56 deformations. The experimental set-up is shown in FIG. 9.

Calibration and Static Force Sensing

The first experiment was performed to characterize the relation between the load cell 62 readings and the forces applied at the tip of the instrument 50. Each joint was tested individually for both antagonistic tendons. A spectra tendon with diameter 0.46 mm and breaking load of about 550N was used to connect a studied link of a joint to an external force sensor in the straightest configuration possible. Once the surgical robot 10 was positioned, the tendons were preloaded at 2N to maintain a degree of stiffness in the instrument's tip. At this stage, the tested joint was actuated to pull the link away from the external force sensor and therefore apply a torque to it. The test was arrested before reaching too high values of tendon tension that could damage the instrument 50.

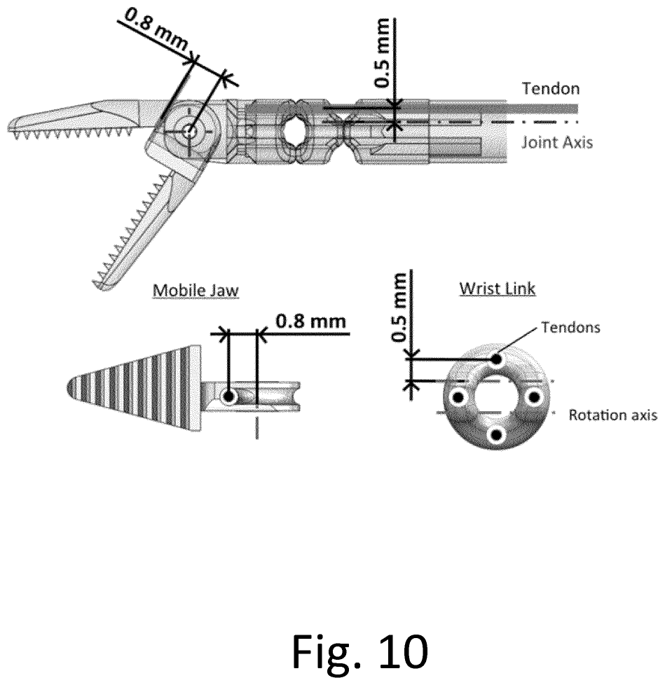

The four tendons needed to actuate the wrist pass at a distance of about 0.5 mm from the wrist joint's rotation axis. This is a very short leverage that acts as a tension amplifier when reading the tendons' tension measured by the load cells 62. For smaller leverage, the force required to actuate the joint is higher; therefore the force reading on the tendon will be increased as the lateral load at the tip of the instruments has a larger cantilever than the cantilever of the tendon. The load cantilever for Joint 1 (wrist joint, first direction) was measured as 10.3 mm, while for Joint 2 (wrist joint, second direction) it was 8 mm. With respect to the grasper test, the load was applied at an approximate distance of 8 mm from the pivot axis of the grasper's jaw, while the actuation tendon had a cantilever of about 0.8 mm with respect to the jaw pivot axis (see FIG. 10).

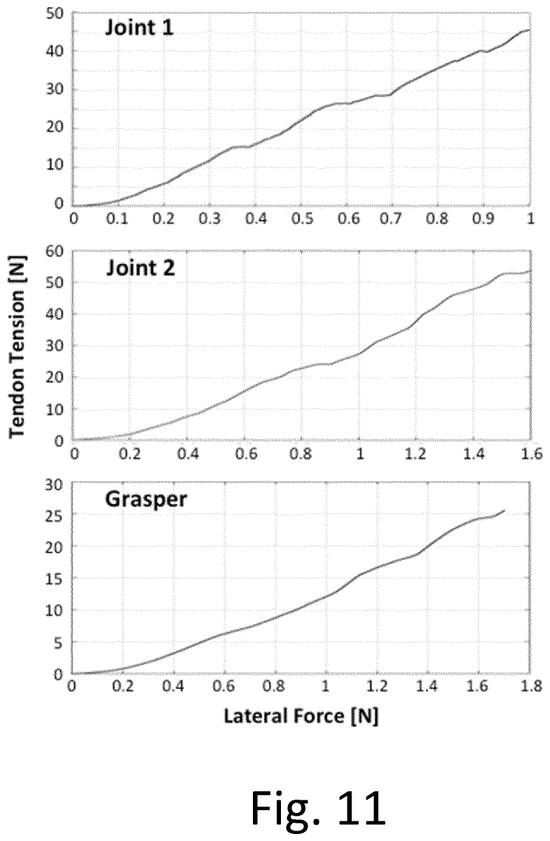

FIG. 11 shows the relationship between the force required to pull the tendons and the force applied by the instrument's tip to the external force sensor. The response of the sensing system, as visible from the graphs, is quite linear for all the three joints. Furthermore, because the response of pairs of antagonistic tendons was very similar, the results of antagonistic pairs were averaged. The analogue signal coming from the load cells 62 was amplified and filtered with a low-pass filter with cut-off frequency of 10 Hz. Consequently, the final residual noise was measured to be about ±0.5N and therefore was negligible for the results. The variations of the measured load with respect to the ideal straight line are due to structural deformation of some elements of the system and also friction. Increasing tendon tensions leads to higher friction between the sliders and their rails. As visible from the graphs related to Joint 1 and Joint 2, the load cell on the tendons of Joint 2 is capable of measuring more lateral force at the tip than in the case of Joint 1. This is due to the fact that, the lateral force on Joint 1 has a larger cantilever with respect to Joint 2. This will cause the tension of the tendons of the first joint to be higher.

Object Grasping with Force Sensing

After calibrating and validating the force measurement, an experiment was designed to test force sensing while actuating the instrument 50 and grasping an object. This was used to validate the functionality of the surgical robot 10. An automated routine was also developed to automatically pretension all the tendons at 2N and then hold the position.

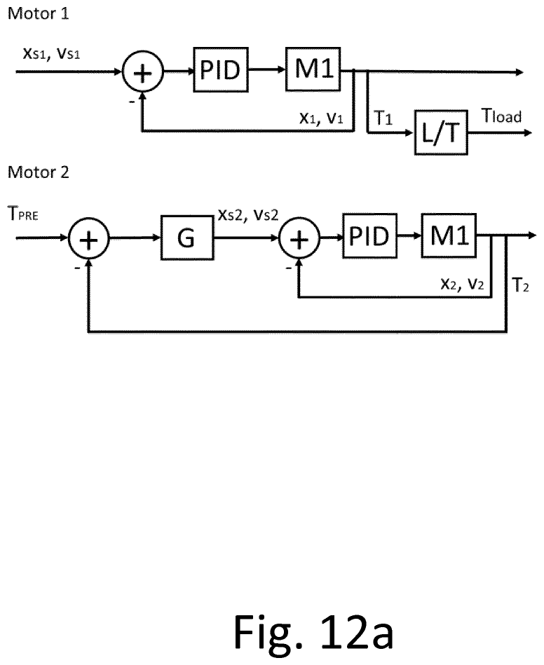

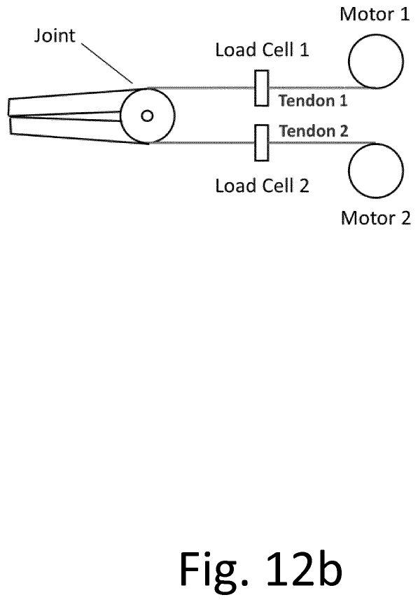

A simple control scheme was devised to control the antagonistic pair of tendons independently with two motors. In order to easily measure the force applied to the end-effector 16 and propagated to its driving tendon, the control had to be decoupled between the two tendons. Therefore, one motor was controlled using a traditional PID loop with position and velocity as set points, while the control for the second motor included the same PID loop with an additional external loop with the objective of maintaining the pretension on the tendon (see FIG. 12).

Xs1 and Vs1 are respectively the position and velocity set points for Motor 1 (M1). These variables are used as an input to control the position of the robotic instrument 50 by the user. The tension on the first tendon is measured by the load cell 62 and converted into the load applied at the tip of the instrument. This is easily done by subtracting the tension of the second tendon from the first one and therefore by scaling by the correct amount found with the first experiment. The second control loop is using the pretension value as input; as a result the motor tries to hold the tension on the second tendon at the pre-set value of 2N.

Therefore, the tension readings from the two branches result to be decoupled, and measuring the lateral load at the tip while controlling the instrument in the space is possible.

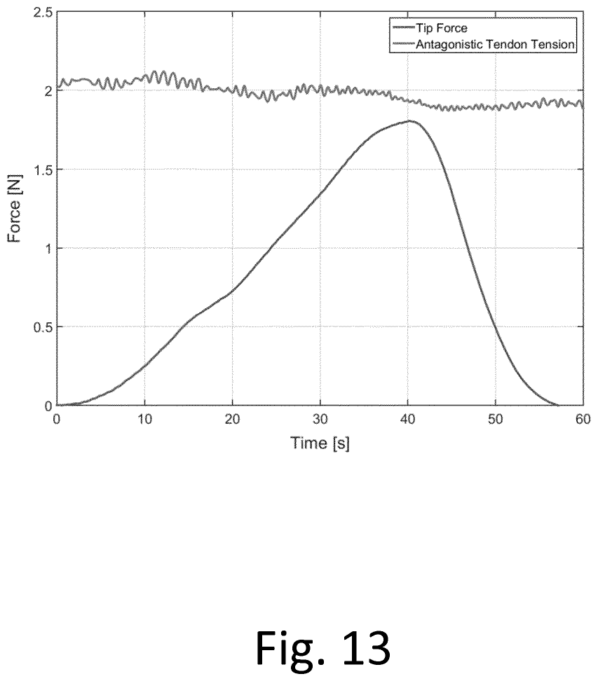

FIG. 13 shows the results from the second experiment. In this experiment the grasper moving jaw was used to pull the spectra tendon connected to the external force sensor, while the second motor was compensated for the tension, trying to keep it constant to the preload value (i.e. 2N). The initial conditions of this experiment were the same as after the automated tensioning routine, therefore the tension on both tendons was equal to 2N. The grasper's jaw was connected to a spectra cable, which was the object to be grasped, that was tied to the external load sensor. This confirmed once more that the transformation between force at the tip and tendon tension was quite linear.

From FIG. 13, it can be seen that the grasping task was 1 minute long and the force measuring on the first tendon showed comparable results to the first experiment. At about 40s the pulling force of the jaw started to be reduced to prevent the tendons from being damaged. The tension on the first tendon is basically the same as the lower line, but scaled up to the tendon tension values. The upper line represents the tension on the second tendon; this clearly fluctuates about 2N, which is the pre-set tensioning and also the set point of the control loops of Motor 2. The controller was using a stability threshold of 0.5N on the tendon tension to prevent the second motor from continuously change direction, due to residual noise. This helped to stabilize the control, although some tension oscillations were still present on the second tendon. As observable at 40s, when the grasper's pulling force starts to decrease, there is also a slight drop on the second tendon's tension. This is due to the design of the controller. When changing direction, Motor 1 is not pulling but releasing the tendon; therefore the load cell on the second tendon measures a drop in tension and actuates Motor 2 in the opposite direction with respect to the previous situation.

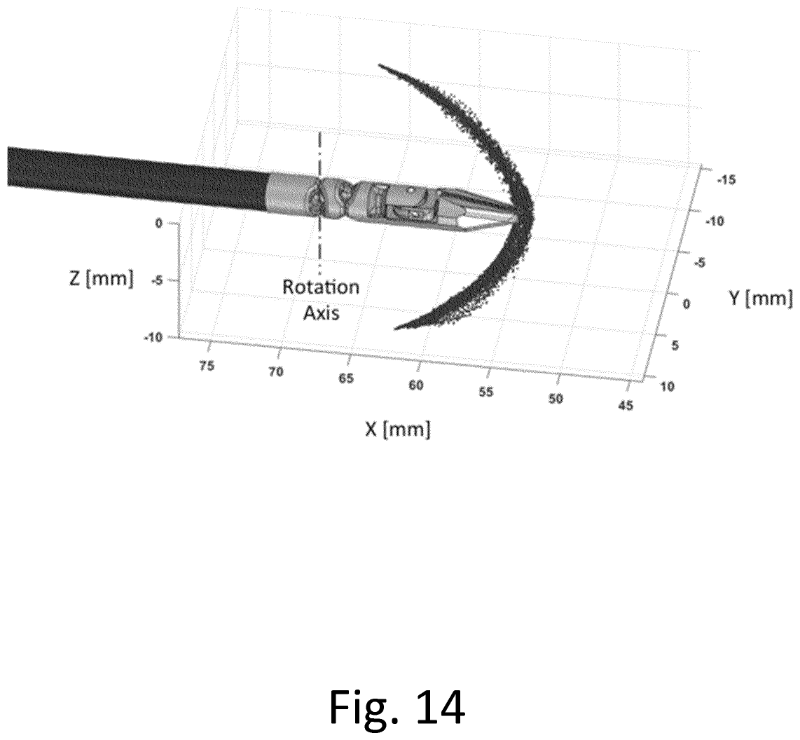

Finally, an experiment to evaluate the repeatability of the position control was carried out. The instrument's tip was moved in the space while actuating the most proximal joint in a cyclic way across the whole ROM. The wrist joint chosen was the one further away from the instrument's tip, since a larger distance introduces higher uncertainty. To track the instrument's tip, an electro-magnetic marker was mounted on the instrument's fixed jaw and tracked with the system trakSTAR (by NDI). It resulted that the deviation in positioning was varying between 1.5 and 3 mm (see FIG. 14), according to where the joint was in the space. In addition to it, the instrument broke after the completion of about 850 motion cycles. Both repeatability and durability tests show promising results for the deployment of rapidly manufactured-disposable instruments in the clinical practice.

Advantages of the Invention

The present invention provides a number of advantages over prior art surgical robots, including:
1. Robot couplings have embedded force sensors to measure the tension applied to each individual tendon.
2. The number of couplings can vary. This example shows a robot with 6 couplings to control three DoF, with tendons actuated by antagonistic pairs. On the other hand the number of couplings could be smaller or larger. In addition to this, the instrument can be designed to control six DoF with six couplings, therefore the couplings would be actuating individually one DoF and would not be paired.
3. The surgical robot is capable of grasping with a known force or limited force when interacting with an object due to the force control.
4. The surgical robot has an intrinsic safety mechanism, since the robot constantly measures the pulling force on each tendon. If a tendon brakes, the robot can react immediately and stop the surgery.
5. The robot couplings are in this example placed in a circular arrangement, but they could equally be placed in any suitable configurations, e.g. linearly.
6. The instrument proximal base clips onto the main body of the surgical robot so it can be quickly and safely inserted. A release mechanism or similar is used to release the surgical instrument.
7. When a surgical instrument is inserted the robot calibrates the instrument by pulling the sliding hooks back and therefore tensioning the tendons at a predetermined pretension value. This is unlike prior art robots where the pretension of the tendons is fixed during the tool assembly. In the present invention, the tension can be varied and can serve a specific purpose.
8. The robot is provided with grooves or cam-like elements that are used to release disengage the sliding hooks from the couplings when the couplings reach their furthest limit.
9. The instrument and the robot body also have embedded electrical contacts for the transmission of signals between the robot and the instrument. When the instrument is plugged onto the robot, the contacts are engaged. This is a useful feature if the surgical instrument has distributed sensors, e.g. force sensors, temperature, pressure, optical sensors etc. Contacts can be placed on the couplings and on the instrument body too.
10. For communication, the instrument carries a NFC (Near Field Communication) chip and is also provided with wireless power transmission.
11. The proximal tool base and tendons separator have one central hole to allow the passage of a tube, electric wire, optical fibres or any additional element that can be of integration to the surgical procedure. For instance a suction/irrigation tube, imaging probes etc. The robot main body also has the same hole at the distal end of the robot.
12. Rotation of the instrument can be 360° free of position limitation.
13. The surgical robot of the present invention has a much smaller footprint compared to prior art robots.

1. A load sensing device for a surgical robot comprising a load sensing means and a coupling mounted to the load sensing means, wherein the coupling is slideable longitudinally and engageable with a tendon for actuating a surgical instrument such that longitudinal movement of the coupling imparts a load on the tendon and the load sensing means measures such load.

2. A load sensing device for a surgical robot according to claim 1, wherein the load sensing device is configured to impart a calibration tension to the tendon upon attachment of a surgical instrument to the surgical robot.

3. A load sensing device for a surgical robot according to claim 2, wherein the load sensing device is configured to monitor the tension of the tendon and restrict actuation of the surgical instrument upon measurement of a tendon tension indicative of the tendon breaking.

4. A load sensing device for a surgical robot according to any of claim 2 or 3 comprising a plurality of load sensing means and an equal number of couplings, wherein measurement of tendon tension is transmitted from each respective load sensing means to a controller and the controller is configured to restrict actuation of the surgical instrument upon measurement of a tendon tension indicative of a respective tendon break.

5. A robotic surgical instrument comprising a rigid hollow shaft positioned between a mounting hub and a surgical tool, wherein the mounting hub has a plurality of couplings slideably mounted thereto, wherein each coupling is associated with a respective tendon passing through the rigid hollow shaft and arranged between said coupling and the surgical tool.

6. A robotic surgical instrument according to claim 5, wherein the mounting hub comprises a domed end and a tendon separator for bringing the tendons into close engagement but maintaining separation.

7. A robotic surgical instrument according to claim 5 or 6, wherein the plurality of couplings are spaced around the mounting hub in a circular configuration.

8. A robotic surgical instrument according to claim 7, wherein the mounting hub is cylindrical and formed of a first part comprising a tendon guide and a second part at least partially enclosing the tendon guide.

9. A surgical robot comprising an attachment interface for actuation of a robotic surgical instrument attachable thereto, wherein the attachment interface comprises means for measuring pulling force applied by the surgical robot to the robotic surgical instrument.

10. A surgical instrument according to claim 9, wherein the attachment interface comprises at least one coupling slideable longitudinally and configured to selectively engage the surgical instrument, wherein said at least one coupling is attached to the means for measuring pulling force and biased to engage the robotic surgical instrument.

11. A surgical robot according to claim 10, wherein the means for measuring pulling force comprise a load sensing means.

12. A surgical robot according to claim 10, wherein the attachment interface comprises a plurality of couplings, wherein each coupling is attached to a respective load sensing means.

13. An attachment interface between a surgical robot and a surgical instrument comprising: a first coupling slideably mounted to a surgical robot and a second coupling mounted to a robotic surgical instrument, wherein the first coupling is movable between a first position in which the first coupling engages the second coupling and prevents longitudinal movement of the robotic surgical instrument relative to the surgical robot and a second position in which the first coupling and the second coupling are disengaged permitting longitudinal movement of the robotic surgical instrument relative to the surgical robot.

14. An attachment interface according to claim 13, wherein the hook comprises a cam feature operable to move the first coupling from a first angular orientation to a second angular orientation when the first coupling is in the second position.

15. An attachment interface according to claim 14, wherein the first coupling is mounted to the surgical robot and is biased in the first angular orientation.

16. An attachment interface according to any of claims 13 to 15, wherein the second coupling is slideably mounted to the surgical instrument such that when the first coupling is in the first position and moved longitudinally relative to the surgical robot, the second coupling is urged longitudinally in the direction of travel of the first coupling.

17. An attachment interface according to any of claims 13 to 16 comprising a plurality of first couplings spaced apart in a circular configuration and an equal number of second couplings, wherein each first coupling is co-operable with a respective second coupling.

18. An attachment interface according to claim 17, wherein the surgical instrument comprises a mounting hub and each second coupling is slideably mounted within a respective groove in the mounting hub.

19. An attachment interface according to claim 18, wherein the surgical robot comprises a mounting plate co-operable with the mounting hub of the surgical instrument, wherein the mounting plate provides an abutment for the mounting hub.

20. An attachment interface according to any of claims 13 to 19, wherein the first coupling is operable to push the second coupling longitudinally towards the robotic surgical instrument.

21. An attachment interface according to any of claims 13 to 19, wherein the first coupling is operable to pull the second coupling longitudinally away from the robotic surgical instrument.

22. An attachment interface according to any of claims 13 to 19, wherein the first coupling is a hook.

23. A surgical robot comprising a body and a mounting interface, wherein the body is rotatable relative to the mounting interface.

24. A surgical robot according to claim 23 further comprising a planetary gear and a pinion wherein the planetary gear is part of the body and the pinion is part of the mounting interface and wherein driving the pinion causes rotation of the body relative to the mounting interface.

25. A surgical robot according to claim 24, wherein the pinion is driven by a motor positioned within the body of the surgical robot.

26. A surgical robot according to any of claims 23 to 25, wherein the body is rotatable through at least three hundred and sixty degrees relative to the attachment portion.

27. A surgical robot according to any of claims 22 to 25, wherein the body and the attachment portion of the surgical robot are cylindrical or flat in geometry.

28. Apparatus for robotic surgery comprising a surgical robot and a robotic surgical instrument, wherein the robotic surgical instrument comprises a surgical tool having at least two degrees of freedom of movement, each degree of freedom of movement being controlled by a respective pair of antagonistic tendons and wherein the surgical robot comprises at least two motors where each motor drives a respective tendon.

29. Apparatus for robotic surgery according to claim 28, wherein the robotic surgical instrument comprises at least three degrees of freedom of movement and six tendons and the surgical robot comprises six motors where each motor drives a respective tendon.

30. Apparatus for robotic surgery according to claim 29 further comprising a seventh motor for rotational translation of the robotic surgical instrument relative to the surgical robot.

31. Apparatus for robotic surgery according to claim 29 or claim 30, wherein each tendon is coupled to a respective motor by an attachment interface between the surgical robot and the robotic surgical instrument, wherein the attachment interface comprises six first couplings on the surgical robot slideable longitudinally to selectively engage respective second couplings on the robotic surgical instrument.

32. Apparatus for robotic surgery according to claim 31, wherein each first coupling is attached to a load cell configured to measure the pulling force applied to the attached first coupling.

33. Apparatus for robotic surgery comprising a surgical robot and a robotic surgical instrument, wherein the robotic surgical instrument comprises a surgical tool having at least two degrees of freedom of movement, each degree of freedom of movement being controlled by a respective pair of antagonistic tendons and wherein the surgical robot comprises at least four motors where each motor drives an antagonistic tendon.

34. Apparatus for robotic surgery according to claim 33, wherein the robotic surgical instrument comprises six degrees of freedom of movement and twelve tendons and the surgical robot comprises six motors where each motor drives a respective tendon.

35. Apparatus for robotic surgery according to claim 34 further comprising a seventh motor for rotational translation of the robotic surgical instrument relative to the surgical robot.

36. Apparatus for robotic surgery according to claim 34 or claim 35, wherein each tendon is coupled to a respective motor by an attachment interface between the surgical robot and the robotic surgical instrument, wherein the attachment interface comprises six first couplings on the surgical robot slideable longitudinally to selectively engage respective second couplings on the robotic surgical instrument.

37. Apparatus for robotic surgery according to claim 33, wherein each first coupling is attached to a load sensing means configured to measure the pulling force applied to the attached first coupling.

38. A method of measuring force applied to a robotic surgical instrument, the method comprising:
i) attaching a robotic surgical instrument having a plurality of tendons to a surgical robot using an attachment interface;
ii) applying a pre-load to each tendon;
iii) actuating the robotic surgical instrument using the tendons and maintaining the pre-load on all non-active tendons; and
iv) measuring and controlling the pulling force applied to each active tendon.

39. A method of measuring force applied to a robotic surgical instrument further comprising the step of:
v) preventing further actuation of the robotic surgical instrument upon measuring a pulling force above a pre-determined threshold or measuring a zero pulling force.

40. A surgical robot comprising a body mounting seven motors therein, wherein each motor is controlled by a respective motor control board provided on a motherboard, wherein each motor control board is modular.

41. A surgical robot according to claim 40, wherein one of the seven motors is configured to enable rotation of the surgical robot around its longitudinal axis.

42. A surgical robot according to claim 41, wherein the surgical robot comprises a planetary gear and a pinion, wherein one of the seven motors drives the pinion to enable rotation of the surgical robot around its longitudinal axis.

43. A surgical robot according to claim 42, wherein at least one of the seven motors drives a lead screw having a coupling and a load cell attached thereto.

44. A surgical robot according to claim 43, wherein six of the seven motors drive respective lead screws with each lead screw having a hook and a load cell attached thereto.

45. A surgical robot according to claim 44 wherein each motor driving a lead screw is attached to a respective lead screw by way of flexible coupling.

46. Apparatus for robotic surgery comprising a surgical robot and a robotic surgical instrument wherein the robotic surgical instrument comprises a near field communication chip for communicating with the surgical robot.

47. Apparatus for robotic surgery according to claim 46, wherein power is transmitted from the surgical robot to the robotic surgical instrument wirelessly.
